(19) United States
(12) Patent Application Publication
(10) Pub. No.: US 2024/0201145 A1
(43) Pub. Date: Jun. 20, 2024

(54) Volatile pH Gradient Mobile Phase Kit

(71) Applicant: Dionex Corporation, Sunnyvale, CA (US)

(72) Inventors: Jung Un Baek, San Jose, CA (US); Shanhua LIEHR, Palo Alto, CA (US); Yuan SUN, Woodinville, WA (US); Christopher A POHL, Union City, CA (US)

(21) Appl. No.: 18/085,179

(22) Filed: Dec. 20, 2022

(51) Int. Cl. G01N 30/34; G01N 30/20; G01N 30/72

(52) U.S. Cl. CPC G01N 30/34; G01N 30/20; G01N 30/72

(57) ABSTRACT

A pH gradient mobile phase preparation kit is described. The preparation kit includes a first component and a second component. The first component includes a first reagent separate from a second reagent. The first reagent, the second reagent, and a solvent together provide a volatile buffer solution having a low pH. The second component includes a base. The base mixed in the solvent provide a volatile base solution having a high pH. A method of separating and detecting one or more analytes comprised in a matrix of components in a sample, using the kit is also described.

### FIELD OF THE INVENTION

This invention is directed to a volatile pH gradient mobile phase kit. In particular, stable mobile phase kits for use with an ion exchange chromatography column coupled to a mass spectrometer are described.

### BACKGROUND OF THE INVENTION

Mass spectrometry (MS) has become widely accepted for selective and sensitive detection in the analysis of large and small molecules. When coupled with ion exchange chromatograph (IEC), atmospheric pressure ionization (API) MS (mostly in the form of electrospray ionization (ESI)) is a powerful tool for identification of compounds and plays a major role in a variety of applications including trace analysis of molecular-mass inorganic and organic anions by IEC-ESI/MS, speciation analysis and metallomics, mass isotopomer measurements of ionic species, organic trace analysis of molecular-mass analytes, glycans and other complex carbohydrates analysis, and proteins. IEC-MS is also useful for analysis of therapeutic monoclonal antibodies (mAb) products, which can be highly heterogeneous due to post-translational modifications and other modifications that occur during manufacture and storage. These modifications often result in charge variants that require salt or pH gradient methods for separation.

A difficulty with using IEC-MS is that conventional salt and pH gradient methods cannot be directly coupled to MS due to the high concentration of salts or other non-volatile mobile phase components. Although several MS compatible mobile phase systems have been recently reported, these mobile phase systems often suffer from poor stability, which leads to inconstant pH gradients and retention time drift of chromatographic peaks.

Accordingly, there remains a need for improved pH gradient mobile phase systems that are effective, stable and compatible for IEC-MS.

### SUMMARY

Systems, methods, and products to address these and other needs are described herein with respect to illustrative, non-limiting, implementations. Various alternatives, modifications and equivalents are possible.

According to a first aspect, a pH gradient mobile phase preparation kit is described. The preparation kit includes a first component and a second component. The first component includes a first reagent separate from a second reagent. The first reagent, the second reagent, and a solvent together provide a volatile buffer solution having a low pH. The second component includes a base. The base combined with the solvent provides a volatile base solution having a high pH.

According to a second aspect, a method of detecting one or more analytes comprised in a matrix of components in a sample is described. The method includes injecting the sample into an injection valve, the injection valve being in fluid communication with a first end of a chromatography column. A volatile buffer solution having a low pH is pumped into the first end of the chromatography column. The volatile buffer solution includes a first reagent and a second reagent provided from a kit. A volatile base solution having a high pH is pumped into the first end of the chromatography column. The volatile base solution includes a base provided from the kit. A proportion of the volatile buffer solution to the volatile base solution in the chromatography column is varied by varying the corresponding amounts of the volatile buffer solution and the volatile base solution that are pumped into the first end of the chromatograph column. This variation of proportions of volatile buffer to volatile base provides a volatile mobile phase with a pH time-gradient flowing through the chromatography column. The method also includes eluting the sample through the chromatographic separation device in the mobile phase thereby separating the analyte from the matrix components. The detection includes injecting the sample into a mass spectrometer to provide a mass spectrum of the one or more analytes.

The pH mobile phase kit and methods described herein provide pH gradient mobile phase systems that are effective, stable and compatible for IEC-MS.

### BRIEF DESCRIPTION OF THE DRAWINGS

The foregoing and other features and advantages of the present embodiments will be more fully understood from the following detailed description of illustrative embodiments taken in conjunction with the accompanying drawings.

FIG. 1 shows a schematic of a chromatography system, according to some implementations.

FIG. 2 is a flow diagram showing steps for separating and detecting one or more analytes comprised in a matrix of components in a sample, according to some implementations.

FIG. 3A illustrates a first kit for providing a volatile pH gradient for a mobile phase, according to some implementations.

FIG. 3B illustrates a second kit for providing a volatile pH gradient for a mobile phase, according to some implementations.

FIG. 3C illustrates a third kit for providing a volatile pH gradient for a mobile phase, according to some implementations.

FIG. 4 shows a chromatogram of Bevacizumab, according to some implementations.

FIG. 5A shows chromatograms of Bevacizumab depicting the effect of degradation of the eluent.

FIG. 5B shows chromatograph of Bevacizumab, according to some implementations.

FIG. 6A illustrates a change in the pH gradient as the eluent degrades.

FIG. 6B shows the stability of a pH gradient of an eluent, according to some implementations.

FIG. 7 is a plot comparing pre-mixing and in-line mixing, according to some implementations.

The figures referred to above are not drawn necessarily to scale, should be understood to provide a representation of particular embodiments, and are merely conceptual in nature and illustrative of the principals involved. The same reference numbers are used in the drawings for similar or identical components and features shown in various alternative embodiments.

### DETAILED DESCRIPTION

In the description of the invention herein, it is understood that a word appearing in the singular encompasses its plural counterpart, and a word appearing in the plural encompasses its singular counterpart, unless implicitly or explicitly understood or stated otherwise. Furthermore, it is understood that for any given component or embodiment described herein, any of the possible candidates or alternatives listed for that component may generally be used individually or in combination with one another, unless implicitly or explicitly understood or stated otherwise. Moreover, it is to be appreciated that the figures, as shown herein, are not necessarily drawn to scale, wherein some of the elements may be drawn merely for clarity of the invention. Also, reference numerals may be repeated among the various figures to show corresponding or analogous elements. Additionally, it will be understood that any list of such candidates or alternatives is merely illustrative, not limiting, unless implicitly or explicitly understood or stated otherwise. In addition, unless otherwise indicated, numbers expressing quantities of ingredients, constituents, reaction conditions and so forth used in the specification and claims are to be understood as being modified by the term “about.”

Accordingly, unless indicated to the contrary, the numerical parameters set forth in the specification and attached claims are approximations that may vary depending upon the desired properties sought to be obtained by the subject matter presented herein. At the very least, and not as an attempt to limit the application of the doctrine of equivalents to the scope of the claims, each numerical parameter should at least be construed in light of the number of reported significant digits and by applying ordinary rounding techniques. Notwithstanding that the numerical ranges and parameters setting forth the broad scope of the subject matter presented herein are approximations, the numerical values set forth in the specific examples are reported as precisely as possible. Any numerical values, however, inherently contain certain errors necessarily resulting from the statistical dispersion found in their respective testing measurements.

Gradient pH mobile phase kits, such as for the analysis of mAb products, are known. For example, U.S. Pat. No. 8,921,113 which is hereby incorporated by reference in its entirety, describes a buffer kit that can generate a linear pH gradient. Such kits often have a first pH buffer solution and a second pH buffer solution. The components can be mixed in various proportions to provide a pH gradient from a low pH to a high pH or from a high pH to a low pH. For example, to elute proteins from a cation exchange resin, a low to high pH gradient is used, while to elute proteins from an anion exchange resin, a high to low pH gradient is used. However, these existing kits include salts that are not volatile and are incompatible with IEC-MS systems. The salts, sometimes with matrix and analyte, such as proteins, tend to precipitate out and can cause clogging, such as in the electrospray ionizing system of the MS. Such clogging can cause pressure buildup, leaks, and poorly controlled flow of eluent through the system. The remedy is costly downtime and maintenance to clean and reset the IEC-MS system into optimal operating condition.

As used herein, matrix materials include non-analyte components that are to be separated from the analyte by chromatography. Analytes are the molecule or compound of interest, and which are detected by the IEC-MS.

In considering the limitations of previous pH gradient kits, it was reasoned that a gradient kit having volatile buffer solutions was needed. However, it was realized that some of the volatile reagents used to make the volatile buffer solutions had a short shelf life after being combined to make the volatile buffers, which presented a challenge to storing and shipping the volatile buffer solution. It is also recognized that temperature can speed up any decomposition processes so that in some implementations, volatile buffer solutions or its components are refrigerated to keep the temperature below about 50° C. during shipping or storage (e.g. below 25° C.). In addition to decomposition altering the chemical properties, such as the pH, which impacts the peak width and separations, decomposition can be accompanied by outgassing and pressurizing of containers which presents a safety hazard.

In contemplating these challenges, it was recognized that the volatile reagents used for making the unstable volatile buffers where stabile when kept separate from each other. It was reasoned that a kit could be designed based on this separation of reagents. According to some implementations of this design, a pH gradient mobile phase preparation kit is described herein. The preparation kit includes a first component and a second component. The first component includes a first reagent separate from a second reagent. The first reagent, the second reagent, and a solvent combined provide a volatile buffer solution having a low pH. The second component includes a base. The base mixed or dissolved in the solvent provides a volatile base solution having a high pH. By keeping the first reagent and the second reagent separate, any interaction that can lead to decomposition of either reagent is eliminated until they are combined or mixed to form the first component. It is understood that this also applies to additional reagents, such as a third, a fourth, and a fifth reagent, which can be kept separate until they are combined with the solvent to form the first component.

In some implementations, the first reagent, the second reagent, the solvent and the base are volatile components. As used herein, volatility refers to the facility of the reagent, the solvent, or base to enter the gas phase. For example, the first reagent, the second reagent, the solvent and the base are considered volatile if they have a vapor pressure of greater than 10 mmHg (1.3 kPa) at room temperature. In some implementations, the first reagent, the second reagent, the solvent and the base are gases at a temperature range from about 25° C. to about 120° C. and at about one atmosphere and at equilibrium, which corresponds to typical conditions for inputting a liquid sample into a MS using atmospheric electrospray. In some implementations, the first component and the second component include only volatile reagents, solvents, and bases, so that an eluent stream made by combination of the first component and the second component only includes volatile reagents, solvents, and bases.

In some implementations, the volatile buffer solution is less stable than the first reagent when separate from the second reagent. That is, the first reagent and the second reagent when kept separate from each other are more stable than when the first reagent and the second reagent are combined or mixed to form the buffer solution. As used herein the “stability” relates to any process, such as chemical decomposition, precipitation, or outgassing, that can change the composition or concentration of one or more of the first reagent and the second reagent. Accordingly, the first reagent and the second reagent each show less than about a 10% (e.g. less than 5%, less than 1%, less than 0.1%) decrease in concentration over a period of a 5 years (e.g., 1 year, 6 months) at room temperature when the first reagent and the second reagent are not combined with each other. In some implementations, the decomposition can be decreased or eliminated by environmental controls, such as by cold storage of the first reagent and the second reagent (e.g., storage below room temperature, or storage below about 10° C.). Mitigation against decomposition can also include maintaining an inert gas in or over the first reagent and the second reagent (e.g., nitrogen sparging and nitrogen headspace in a sealed container). In some implementations, the buffer solution shows less than a 10% (e.g., less than 5%, less than 1%, less than 0.1%) concentration change in any one of the first reagent, the second reagent, and the solvent over 10 days (e.g., over 5 days). The kit can be shipped and stored for a storage time (e.g., a year) until the first reagent and the second reagent are needed to form the buffer solution. The buffer solution can then be prepared and used for a time (e.g., 2 weeks, 1 week, or 1 day) that is shorter than the storage time but still long enough for use with IEC-MS.

In some implementations, combining the first component with the second component in varied proportions provides a volatile mobile phase having a pH in a range between the low pH and the high pH. For example, the low pH is higher than about 4 (e.g., higher than about 5, higher than about 6, or higher than about 7) and the high pH is lower than about 11 (e.g., lower than about 10, lower than about 9, or lower than about 8).

In some implementations, the first reagent is provided in an undiluted form and the second reagent is provided in a form diluted by the solvent. As used here, the undiluted form refers to the need to add the solvent to provide the desired concentration for use as the volatile buffer. For example, the first reagent in an undiluted form can be a neat (e.g., >99% pure) form of a chemical compound. In some implementations, the first reagent or the second reagent are provided as a concentrate including a portion of the total solvent required to provide the desired concentration for use as the volatile buffer.

In some implementations, the second reagent is added to the first reagent to form the volatile buffer solution. For example, the second reagent dissolved in the solvent is added to the first reagent, which is a concentrate or in a neat form, to provide the volatile buffer solution. In some implementations, the first reagent is added to the second reagent to form the volatile buffer solution. For example, the first reagent is a concentrate, or a neat form and it is added to the second reagent, which is dissolved in the solvent, to form the volatile buffer solution. In some implementations, one or both of the first reagent and the second reagent are provided in an undiluted form, and the solvent is combined with the first reagent and the second reagent in any order to form the volatile buffer solution. In some implementations, the first reagent is diluted with a first portion of the solvent and the second reagent is diluted with a second portion of the solvent and these are combined to provide the volatile buffer solution.

In some implementations, the base is provided in an undiluted form. For example, the base can be provided as a crystalline, powdered, or liquid pure compound that can be dissolved in the solvent to provide the volatile base solution. Alternatively, the base is provided as a concentrate in a liquid where the concentrate is diluted with the solvent to provide the volatile base solution. In some implementations, the base is provided already diluted in the solvent and is used without further dilution to provide the volatile base solution.

Any volatile base can be used in forming the second component. In some implementations, the base comprised in the second component is ammonia or an amine. For example, the base can be an ammonium hydroxide solution that is used without further dilution to provide the volatile base solution. In an alternative example, the base can be an ammonium hydroxide concentrate that can be diluted with the solvent to provide the volatile base solution. The amine can be used as a neat amine that is dissolved in the solvent to provide the volatile base solution. The amine can alternatively be provided as a concentrate dissolved in the solvent but requiring dilution with the solvent prior to use as the volatile base solution. The amine can also be provided as a solution in the solvent that can be used without further dilution as the volatile base solution. The amine can be provided in the protonated form with a hydroxyl counter ion. In some implementations, the concentration of the base in the volatile base solution is between about 1 mM (e.g., about 5 mM, about 9 mM) and about 100 mM (e.g., about 50 mM, about 15 mM).

In some implementation, the base is selected from, ammonia, hydrazine, methylamine, di-methylamine, trimethylamine, ethylamine, diethylamine, triethylamine, isobutyl amine, dibutyl amine, N,N-diisopropylethylamine, morpholine, piperazine, ethylenediamine, 1,4-diazabicyclo[2.2.2]octane, and mixtures thereof. In some implementations, the base is ammonia in water (i.e., ammonium hydroxide).

In some implementations, the first reagent used in providing the buffer solution includes ammonia or an amine. For example, the first reagent can be selected to include one or more of ammonia, hydrazine, methylamine, di-methylamine, trimethylamine, ethylamine, diethylamine, triethylamine, isobutyl amine, dibutyl amine, N,N-diisopropylethylamine, morpholine, piperazine, ethylenediamine, and 1,4-diazabicyclo[2.2.2]octane. In some implementations, the first reagent includes a volatile counter ion. For example, in some implementations, the first reagent is a carbonate salt. In some implementations, the first reagent is ammonium bicarbonate. In some implementations, the first reagent is an amine carbonate such as trimethylammonium carbonate. In some implementations, the amine or amine salt can be used as the neat compound (e.g. a powered, crystalline salt, or liquid) that is dissolved in the solvent to provide volatile buffer solution with the second reagent. In some implementations, the amine or amine salt is provided as a concentrate diluted in the solvent but requiring additional dilution with the solvent to the volatile buffer solution with the second reagent. In some other implementations, the amine or amine salt can be provided as a solution in the solvent and can be used without further dilution as the volatile buffer solution in combination with the second reagent.

In some implementations, the second reagent used in providing the buffer solution is a carboxylic acid. For example, a mono-carboxylic acid. In some implementations, the carboxylic acid is selected from one or more of formic acid, acetic acid, trifluoracetic acid, difluoroacetic acid, propionic acid, butyric acid, valeric acid and positional isomers thereof. In some implementations, the carboxylic acid is acetic acid. In some implementations, the carboxylic acid is provided as a neat carboxylic acid which is dissolved in the solvent to form the buffer solution with the first reagent. In some implementations, the carboxylic acid is provided as a concentrate diluted in the solvent but requiring additional dilution with the solvent prior to use to form the buffer solution with the first reagent. Alternatively, in some implementations, the carboxylic acid is provided as a solution in the solvent that can be used without further dilution to form the buffer solution with the first reagent. In some implementations, the concentration of the carboxylic acid in the volatile base solution is between about 1 mM (e.g., about 10 mM, about 20 mM) and about 100 mM (e.g., about 75 mM, about 50) mM).

In some implementations the volatile buffer solution and the volatile base solution does not include halide salts, phosphate salts, sulfate salts, nitrate salts or their corresponding acids. In some implementations the volatile buffer solution and the volatile base solution do not include molecules with phosphate, nitrate, or sulfate functional groups. In some implementations the volatile buffer solution and the volatile base solution do not include metal ions such as ions of lithium, sodium, potassium, magnesium, cesium or the transition metals. As used herein, not including a molecule or salt means that the component is not added deliberately and is present below about 5 ppm (e.g., less than 1 ppm, less than. 1 ppb, less than 10 ppm, less than 1 ppb).

In some implementations, the kit includes a first container for containing the first reagent, a second container containing the second reagent, and a third container containing the second component. The first container, the second container, and the third container can be substantially gas impermeable and gas tight. For example, the first container, the second container, and the third container all have a carbon dioxide gas permeability less than about 2000 cc.mm/m²-24 hr.Bar (e.g. less than about 1000, less than about 500, less than about 250). In some implementations, the second container and the third container have a carbon dioxide gas permeability greater than about 50 cc.mm/m²-24 hr (e.g. greater than about 100, greater than about 200). In some implementations, the first container has a carbon dioxide gas permeability less than about 225 cc.mm/m²-24 hr. Bar (e.g., less than about 200, less than about 150, less than about 100, less than about 50). In some implementations, the first container has a carbon dioxide gas permeability greater than about 1cc.mm/m²-24 hr (e.g. greater than about 10, greater than about 20, greater than about 30, greater than about 40).

In some implementations, the first container includes one or more of PETG (Polyethylene terephthalate glycol), PMMA (Poly(methyl methacrylate), or ETFE (Ethylene tetrafluoroethylene) in its material composition. In some implementations, the first container includes PETG in its material composition. In some implementations, the second container includes one or more of LDPE (low density polyethylene), HDPE (high density polyethylene), PP (polypropylene), PPCO (polypropylene copolymer), PMP (polymethylpentene), FLPE (fluorinated high density polyethylene), FEP (fluorinated ethylene propylene), PFA (perfluoroalkoxy alkanes), ETFE (ethylene tetrafluoroethylene), PSF (polysulfone), PC (Polycarbonate) in its material composition. In some implementations the second container includes HDPE in its material composition. In some implementations, the third container includes one or more of LDPE, HDPE, PP, PPCA, PMP, FEP, PFA, and ETFE in its material composition. In some implementations the third container include HDPE in its material composition.

In some implementations, a cap or cover that is substantially gas impermeable and closes on the container is included to provide a gas tight seal.

In some implementations, one or more of the containers (e.g., the first, the second and the third container) include a cap that can be directly coupled to the HPLC mobile phase line (e.g., having adaptor ports that can couple to tubing). In some implementations, one or more of the containers is a flexible container such as a bag, pouch, or carton. In some implementations, one or more of the containers is a bottle, such as an HDPE bottle or a PETG bottle. In some implementations, one or more of the containers is a vial or microtube. In some implementations, one or more of the containers is a syringe. In some implementations, one or more of the containers is an ampule.

One or more of the first reagent, the second reagent and the base can be contained by their respective containers in an inert atmosphere. For example, reagents and any solvent can be sparged with an inert gas such as nitrogen or argon and sealed. In some implementations, any headspace of the container is of an inert gas.

In some implementations, the solvent includes water, such as an ultrapure water. For example, the water can be an HPLC grade water (e.g., ASTM Type 1 water) having high resistivity (>18 megohms at 25° C.), low TOC (<5 ppb), low silica (<3 ppb), low sodium (<1 ppb) and low chloride (<1 ppb). In some implementations, the water is an MS grade water, such as PIERCE™ Water, LC-MS Grade water available from Thermo Fisher Scientific (Waltham, MA) cat. No. 51140. In some implementations, the solvent also includes less than about 10% of a water miscible compound or co-solvent. For example, the miscible compound can be a polar solvent such as a polar solvent selected from acetonitrile, tetrahydrofuran (THF), acetone, dimethylsulfoxide (DMSO), dimethylformamide (DMF), methanol, ethanol, propanol, and mixtures thereof. The co-solvent can also have a high purity, such as an HPLC or MS grade co-solvent. In some implementations, a co-solvent or a mixture of water and the co-solvent is used to provide a concentrate of the first reagent, the second reagent or the base, and water or a mixture of water and a co-solvent are used to further dilute the concentrate to provide the volatile buffer solution or the volatile base solution, respectively. In some implementations, water is used to provide a concentrate of the first reagent, the second reagent or the base, and water or a mixture of water and co-solvent are used to further dilute the first reagent, the second reagent or the base to provide the volatile buffer solution or the volatile base solution respectively.

In addition to miscibility, the solvent and co-solvent are chosen so that the solvent and co-solvent are inert to the analyte, such as not causing the denaturing of an analyte such as a protein. The solvent and co-solvent also should not cause any of the analyte, or components in the volatile buffer solution or volatile base solution to precipitate. In some implementations, the solvent and co-solvent are inert to the first container, the second container, and the third container, such that the container material is not dissolved by the solvent or co-solvent, or the solvent or co-solvent is not decomposed (e.g. polymerized) by contact with the container material.

In some implementations, the solvent is provided with the kit, and no additional solvent or co-solvent is needed to prepare the volatile buffer solution or the volatile base solution. For example, the solvent can be included with one or more of the first reagent, with the second reagent, or with the base in the kit. In some implementations, the solvent is provided mixed with the first reagent, the second reagent, or the base. In some other implementations, the solvent is provided in the kit but separate (e.g., in a separate container) from the first reagent, the second reagent, or the base. In some implementations, one or more of the first reagent, the second reagent or the base is mixed with the solvent as provided in the kit (e.g., as a concentrate), and additional separate solvent is also provided in the kit for mixing with one or more of the first reagent, the second reagent, or the base.

In some implementations, a first portion of a total amount of solvent required to make the volatile buffer solution or volatile base solution is included in the kit, where a second portion of the total amount of solvent required is not included in the kit and is added by the user of the kit to prepare the volatile buffer solution or the volatile base solution. In such an implementation, the first portion of the total amount of solvent and the second portion of the total amount of solvent can have different compositions. For example, the first portion of the total amount of solvent can include only solvent, while the second portion of the total amount of solvent can include the solvent and the co-solvent: or the first portion of the total amount of solvent can include solvent and co-solvent, while the second portion of the total amount solvent can include only the solvent.

In addition to containers for the first reagent, the second reagent, base, and solvent, the kit can include other items for convenience and use. In some implementations, the kit includes a case or box to hold the containers or other elements of the kit. In some implementations, the kit includes instructions on how to combine/mix and use the first reagent, the second reagent, base, and solvent. The instructions can be provided as a hard copy, such as on a written sheet, on the case or box, on a computer memory device (e.g., a thumb drive, a disk) or in an instruction manual. The instructions can also be on a website where the address, a QCR code, or a bar code is provided as a written instruction in or on the kit to access to the website. The kit can also include additional containers for mixing, stirrers, pipettes, syringes, and the like for handling the chemical components. In some implementations, the reagents do not require any additional equipment such as an analytical balance and labware for use. For example, the first reagent, the second reagent, the base and the solvent can be combined directly, without any weighing or measuring. The kit can also include bags and wipes for disposal and cleanup after use of the kit. In some implementations, no waste of the first reagent, the second reagent, the base or the solvent is generated in preparing the volatile buffer solution or volatile base solution since all of these are used in preparing the solutions.

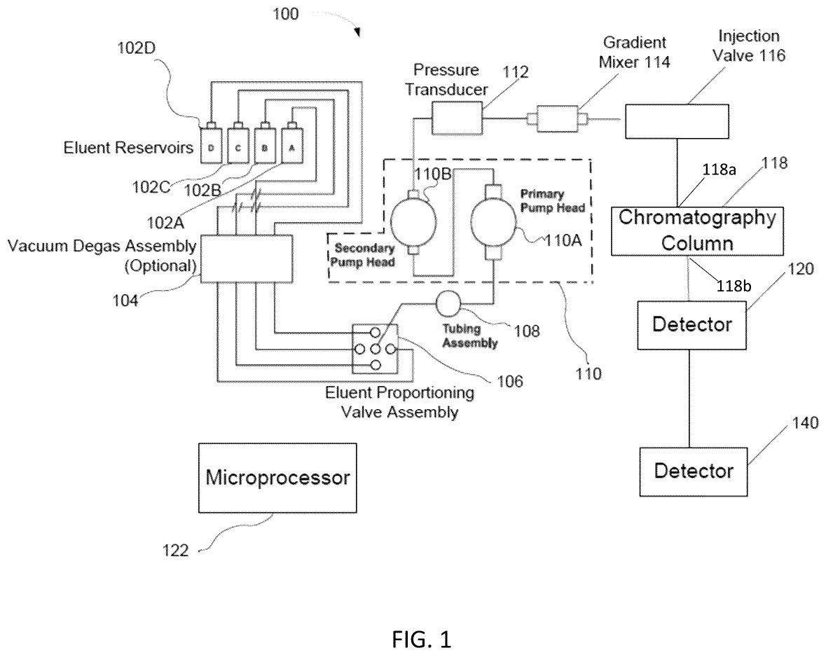

FIG. 1 illustrates a schematic of a chromatography system 100 configured to perform an isocratic elution or a gradient elution with up to four different mobile phase types, according to some implementations. Chromatography system 100 can include four mobile phase reservoirs (102A, 102B, 102C, 102D), an optional degas assembly 104, an eluent proportioning valve assembly 106, a tubing assembly 108, a pump 110, a pressure transducer 112, a gradient mixer 114, an injection valve 116, a chromatography column 118, a detector 120, another detector 140, and a microprocessor 122.

Pump 110 can be configured to pump a liquid from one or more reservoirs through system 100. The pumped liquid may flow through an optional degas assembly 104, and then to eluent proportioning valve assembly 106. A predetermined proportion of liquid can be extracted from each of the four mobile phase reservoirs (102A, 102B, 102C, 102D), or a subset thereof (e.g. 102A and 102B, or 102A, 102B and 102C) using eluent proportioning valve assembly 106 and transmitted to tubing assembly 108 and then pump 110. Pump 110 includes a primary pump head 110A and a secondary pump head 110B. The eluent proportioning valve assembly 106 can direct pump 110 to draw on one of the four mobile phase reservoirs for a predetermined time period and then switch to another mobile phase reservoir. Typically, the pump will draw upon each of the selected mobile phase types at least once during a piston cycle to form a plurality of adjoining solvent volumes. For example, four mobile phase reservoirs (102A, 102B, 102C, 102D), or a subset thereof, can be used for the pH gradient elution. This will initially form a heterogeneous solvent volume (unmixed) containing liquid volume A, liquid volume B, liquid volume C, and liquid volume D. Note that solvent volumes A, B, C, or D can be referred to as a plug of liquid that flows through a conduit such that there is not complete homogenization between the four plugs. Solvent volumes A, B, C, or D can be in an adjoining and serial relationship. The proportion of solvent volumes A, B, C, or D depends on the timing in which eluent proportioning valve assembly 106 draws on a particular reservoir. The heterogeneous solvent volume is outputted from pump 110 and corresponds to an outputted solvent from one pump cycle. Subsequent to pump 110, the heterogeneous solvent volume can be mixed in gradient mixer 114. Although pump 110 is shown as a ternary pump configured to be in a low-pressure gradient format, the pump could also be a binary pump configured to be in a high pressure gradient format.

In some implementations, two mobile phase reservoirs such as mobile phase reservoirs 102A and 102B, can be used for the volatile buffer solution and the volatile base solution, respectively, for a gradient elution while mobile phase reservoirs 102C and 102D are not used. In some implementations only two reservoirs, such as mobile phase reservoirs 102A and 102B, are included in chromatography system 100, where mobile phase reservoirs 102C and 102D and associated tubing, connections, etc. are not included in the chromatography system 100. The proportion of solvent volumes A and B can change with time to form a pH gradient elution.

In some implementations, three mobile phase reservoirs, such as mobile phase reservoirs 102A, 102B and 102C are used. For example, mobile phase reservoir 102A is charged with the first reagent (e.g. ammonium carbonate), the mobile phase reservoir 102B is charged with the second reagent (e.g. acetic acid), and the mobile phase reservoir 102C is charged with the base (e.g. ammonium hydroxide).

The output of pump 110 serially flows the eluent to pressure transducer 112, gradient mixer 114, injection valve 116, a first end 118a of the chromatography column 118, detector 120 (optional), and then to another detector 140. Pressure transducer 112 can be used to measure the system pressure of the mobile phase being pumped by pump 110. Injection valve 116 can be used to inject an aliquot of a sample into an eluent stream. Chromatography column 118 can be used to separate various matrix components present in the liquid sample from the analytes of interest. An output of chromatography column 118 can be fluidically connected to detector 120, and then to another detector 140. Detectors 120 and 140 can be in the form of an ultraviolet-visible spectrophotometer (UV) to monitor an absorbance of incident light at a predetermined wavelength as a function of time, a fluorescence detector (FLD), evaporative light scattering detector, multiangle light scattering detector (MALS), a mass spectrometer, and a combination thereof. MALS is a technique for measuring the light scattered by a compound into a multitude of angles. MALS can provide a determination of the molar mass and the average size of molecules in solution, by detecting how the compounds scatter light.

In some implementations, a suppressor (not shown) can also be included. The suppressor can remove any non-volatile salts that might be in the eluent such as that are present in the matrix of the sample or contaminants in the solvent. For example, the suppressor can be placed in series before at least one of detector 120 and detector 140.

In one way of operation of the chromatography system 100, a non-destructive detector can be used in an upstream detector 120 such as ultraviolet-visible spectrophotometer or fluorescence detector to identify an analyte such as a mAb. Next, the sample can then be inputted into another detector 140 (downstream of detector 120), which can be a destructive detector such as MS to further characterize the sample. In some implementations, only a MS detector is used.

In some implementations, a split stream is used to direct a first portion of the eluent and sample from a second 118b end of the chromatography column 118 to detector 120 and detector 140 in parallel (not shown) rather than in series. Such an arrangement is useful where both detectors are destructive detectors such as a CAD (Charged Aerosol Detector), an ELSD (Evaporative Light-Scattering Detector), and an MS detector.

Chromatography column 118 can separate one or more analytes of a sample that is outputted at different retention times. For example, in some implementations, the chromatography column 118 can be in the form of a cation exchange column (with either weak or strong cation exchange sites). The resin inside a column can include a substrate, a coating on the substrate, and a grafted cation exchange group attached to the coating. The substrate can be a crosslinked copolymer of ethylvinylbenzene and divinylbenzene. The coating can be a neutral hydrophilic polymer on a surface of the substrate. The grafted cation exchange group can be carboxylate or a sulfonate. Commercially available cation exchange columns that can be suitable for use with the methods described herein include Thermo Scientific™ ProPac™ WCX-10, Thermo Scientific™ ProPac™ Elite WCX, Thermo Scientific™ MAbPac™ SCX-10, Thermo Scientific™, Thermo Scientific™ ProSwift™ SCX-IS, YMC BioPro IEX SP, YMC BioPro IEX SF, Waters™ BioResolve™ SCX mAb, Sepax Proteomix™ WCX, Sepax Proteomix® SCX, and Agilent™ Bio Mab.

In some implementations, chromatography column 118 can be in the form of an anion exchange column (with either weak or strong anion exchange sites). The resin inside a column can include a substrate, a coating on the substrate, and a grafted anion exchange group attached to the coating. The substrate can be a crosslinked copolymer of ethylvinylbenzene and divinylbenzene. The coating can be a neutral hydrophilic polymer on a surface of the substrate. The grafted anion exchange group can be quaternary ammonium groups (or tertiary amine groups) attached to the coating. A commercially available anion exchange column that can be suitable for use with the methods described herein is the Thermo Scientific™ ProPac™ SAX column (strong anion exchange, 10 micron diameter particle size), Thermo Scientific™ DNAPac™ PA100 column, Thermo Scientific™ DNAPac™ PA200 column (non-porous substrate particle with quaternary ammonium functionalized nanobeads), Dionex™ Ionpac™ AS32-Fast column (supermacroporous ethylvinylbenzene polymer cross-linked with divinylbenzene having alkanol quaternary ammonium groups), Thermo Scientific™ ProSwift™ SAX-IS column, Thermo Scientific™ DNASwift™ SAX-IS Oligonucleotide column, YMC BioPro™ IEX QA column (hydrophilic porous polymer beads with quaternary ammonium groups), YMC BioPro™ IEX QF column (hydrophilic non-porous polymer beads with quaternary ammonium groups), and Waters Protein-Pak Hi Res Q (non-porous polymethacrylate particle substrate).

Microprocessor 122 can include a memory portion and be used to control the operation of chromatography system 100. Microprocessor 122 may either be integrated into chromatography system 100 or be part of a personal computer that sends a signal to communicate with chromatography system 100. Microprocessor 122 may be configured to communicate with and control one or more components of chromatography system such as pump 110, eluent proportioning valve 106, injection valve 116, and detectors 120 and 140. Memory portion can include software or firmware instructions on how to control pump 110, eluent proportioning valve 106, injection valve 116, and detectors 120 and 140.

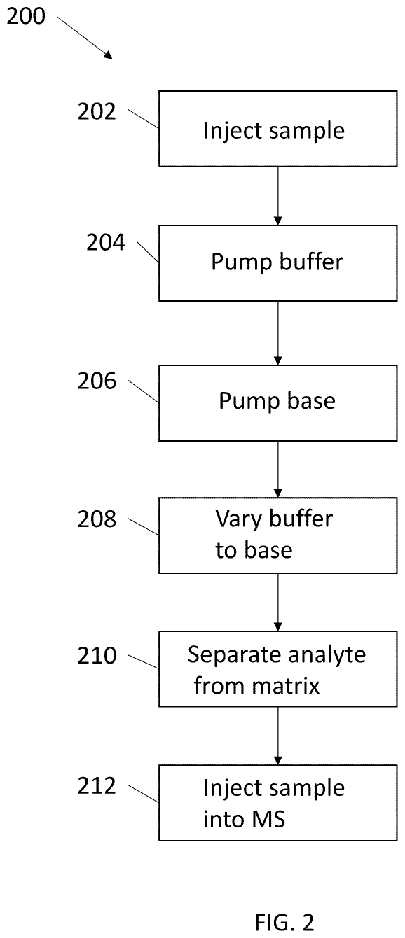

The chromatograph system 100 or a similar system can be used with the pH gradient mobile phase preparation kit described herein. FIG. 2 is a flow diagram depicting how the chromatography system 100 (FIG. 1) or similar systems can be used in a method 200 of separating and detecting one or more analytes comprised in a matrix of components in a sample, according to some implementations.

The method 200 includes step 202 of injecting the sample into the injection valve 116. The valve 116 is in fluid communication with the first end 118a of the chromatograph column 118.

The volatile buffer solution, having a low pH, is pumped in step 204 by pump 110 from one of the mobile phase reservoirs (102A, 102B, 102C or 102D) into the first end of the chromatograph column 118. In some implementations, the volatile buffer solution is prepared as a mixture of the first reagent and the second reagent which is charged in one of the mobile phase reservoirs. For example, the first reagent and the second reagent can be mixed together in the mobile phase reservoir 102A or mixed together before placement into the mobile phase reservoir 102A.

Step 206 includes pumping the volatile base solution having a high pH by pump 110 from one of the eluent reservoirs, not used by the volatile buffer solution, into the first end 118a of the chromatograph column 118. The volatile base solution can include the base in water (e.g., ammonium hydroxide).

The method further includes step 208 which it to vary the proportions of the volatile buffer solution to the volatile base solution in the chromatograph column. This is accomplished by varying the corresponding amounts of the volatile buffer solution and the volatile base solution that are pumped to the first end 118a of the chromatography column 118.

The sample is eluted through the chromatography column 118 carried in the volatile mobile phase thereby separating the analyte from the matrix components, as indicted in step 210. In some implementations where more than one analyte is present, one or more analytes can be separated from other analytes present and independently detected.

In step 212, the analyte and mobile phase are injected into a mass spectrometer, such as detector 140. In some implementations, the mass spectrometer includes an electrospray ionization source to create an aerosol of the analyte and volatile mobile phase.

In some implementations, the first reagent and the second reagent from the kit are combined in-line to form the volatile buffer solution. In such implementations, the first reagent and the second reagent are in a solution form, such as diluted with the solvent. For example, the first reagent is contained in a first reservoir (not shown in FIG. 1) that is fed at a first flow rate to mobile phase reservoir 102A, and the second reagent is contained in a second reservoir (not shown in FIG. 1) that is fed at a second rate to mobile phase reservoir 102A. The first flow rate and the second flow rate are selected to provide the correct proportion of the first reagent and second reagent to provide the volatile buffer solution in the mobile phase reservoir 102A, which is then pumped to the first end of the chromatography column through the intervening elements such the eluent proportion valve assembly 106, gradient mixer 114 and associated pump 110, tubing and pressure transducer 112 (FIG. 1). In an alternative implementation, the first reagent and the second reagent, both in solution form, are contained in two separate reservoirs, such as 102A and 102B. In such an implementation, the eluent proportion valve assembly 106, gradient mixer 114 and associated pump 110, tubing and pressure transducer 112, mix the components to provide the buffer solution directly with the volatile base solution.

In some implementations, the first reagent and the second reagent, provided in the kit, are combined to form the volatile buffer solution less than 10 days prior to pumping the volatile buffer solution into the first end of the chromatograph column. In some implementations, the first reagent and second reagent are mixed less than 5 days prior to pumping. In some implementations, the first reagent and second reagent are mixed less than 24 hours prior to pumping.

In some implementations, the chromatograph column is an ion exchange column. For example, the chromatograph column can be a cation exchange column, or the chromatograph column can be an anion exchange column.

In some implementations, the analyte is a molecule having an isoelectric point (pI) between the low pH and the high pH. For example, a biomolecule with a pI between pH 4 and pH 10. In some implementations, the molecule can be a protein. For example, and without limitation, the protein can include a peptide, polypeptides, antibodies, enzymes, and capsids. The proteins can be synthetic or from natural sources such as cells and viruses. The analyte can also be a high molecular weight charged molecule (e.g., >1 kDa), such as charged polysaccharides. In some implementations, the analyte is a synthetic macromolecule, such as an anionic polymer or a cationic polymer. In some implementations, the analyte includes a small molecule, such as having a low molecular weight (e.g., <1 kDa). Some examples of small molecules include sugars, amino acids, and metabolites. In some implementations, mixtures of the above-mentioned analysts are included as a sample, such as large biomolecules and small molecules.

In some implementations, the pH time-gradient includes a positive gradient, wherein the pH increases with time while separating the analyte from the matrix components. In some implementations, the pH time-gradient includes a negative gradient, wherein the pH decreases with time while separating the analyte from the matrix components. In some implementations, the pH time-gradient includes a negative gradient, and a positive time gradient while separating the analyte from the matrix components.

The following numbered paragraphs 1-22 provide some examples of the embodiments disclosed herein.

Paragraph 1. A pH gradient mobile phase preparation kit comprising: a first component comprising a first reagent separate from a second reagent, wherein the first reagent, the second reagent, and a solvent together provide a volatile buffer solution having a low pH: and a second component comprising a base, wherein the base combined with the solvent provide a volatile base solution having a high pH.

Paragraph 2. The kit according to paragraph 1, wherein the first reagent, the second reagent, the solvent and the base are volatile components.

Paragraph 3. The kit according to paragraph 1 or paragraph 2, wherein the volatile buffer solution is less stable than the first reagent separate from the second reagent.

Paragraph 4. The kit according to any of paragraphs 1-3, wherein combining the first component with the second component in varied proportions provides a volatile mobile phase having a pH in a range between the low pH and the high pH.

Paragraph 5. The kit according to any of paragraphs 1-4, wherein the first reagent is provided in an undiluted form and the second reagent is provided in a form diluted by the solvent.

Paragraph 6. The kit according to any of paragraphs 1-5, wherein the first reagent is provided in an undiluted form and the second reagent is provided in an undiluted form, and the solvent is added to form the volatile buffer solution.

Paragraph 7. The kit according to any of paragraphs 1-6, wherein the base is provided in an undiluted form.

Paragraph 8. The kit according to any of paragraphs 1-7, wherein the base is ammonia or an amine.

Paragraph 9. The kit according to any of paragraphs 1-8, wherein the second reagent is a carboxylic acid.

Paragraph 10. The kit according to any of paragraphs 1-8, wherein the first reagent includes ammonia or an amine.

Paragraph 11. The kit according to paragraph 10, wherein the first reagent is ammonium bicarbonate.

Paragraph 12. The kit according to any one of paragraphs 1-11, including a first container containing the first reagent, a second container containing the second reagent, and a third container containing the second component, wherein the first container, the second container, and the third container are substantially gas impermeable and gas tight.

Paragraph 13. The kit according to paragraph 12, wherein the first container has a carbon dioxide gas permeability of less than 225 cc.-mm/m²-24 hr.-Bar.

Paragraph 14. The kit according to any one of paragraphs 1-13, wherein the solvent includes water.

Paragraph 15. A method of detecting one or more analytes comprised in a matrix of components in a sample, the method comprising: injecting the sample into an injection valve, the injection valve being in fluid communication with a first end of a chromatography column: pumping a volatile buffer solution having a low pH into the first end of the chromatography column, the volatile buffer solution comprising a first reagent and a second reagent provided from a kit: pumping a volatile base solution having a high pH into the first end of the chromatography column, the volatile base solution including a base provided in the kit: varying a proportion of the volatile buffer solution to the volatile base solution in the chromatography column by varying the corresponding amounts of the volatile buffer solution and the volatile base solution pumped to the first end of the chromatography column, thereby providing a volatile mobile phase with a pH time-gradient flowing through the chromatography column: eluting the sample through the chromatography column in the volatile mobile phase thereby separating the analyte from the matrix components: injecting the sample into a mass spectrometer to provide a mass spectrum of the one or more analytes.

Paragraph 16. The method according to paragraph 15, wherein the first reagent and the second reagent from the kit are combined to form the volatile buffer solution less than 10 days prior to pumping the volatile buffer solution into the first end of the chromatograph column.

Paragraph 17. The method according to paragraph 15 or paragraph 16, wherein the mass spectrometer includes an electrospray ionization source to create an aerosol of the analyte and the volatile mobile phase.

Paragraph 18. The method according to any of one of paragraphs 15-17, wherein the first reagent includes ammonium bicarbonate, and the second reagent includes acetic acid.

Paragraph 19. The method according to any one of paragraphs 15-18, wherein the chromatograph column is an ion exchange column.

Paragraph 20. The method according to any one of paragraphs 15-19, wherein the analyte is a biomolecule having a pI between pH 4 and pH 10.

Paragraph 21. The method according to any one of paragraphs 15-20, wherein the pH time-gradient includes a positive gradient, wherein the pH increases with time while separating the analyte from the matrix components.

Paragraph 22. The method according to any one of paragraphs 15-21, wherein the first reagent and the second reagent from the kit are combined in-line to form the volatile buffer solution.

The kit can be used as described to provide pH gradient eluents that can mitigate damage or down time due to injector systems clogging of a MS coupled to IEC. For illustrative purposes, and without limiting the uses, some further examples are discussed below.

### EXEMPLIFICATIONS

### Introduction

Therapeutic monoclonal antibody (mAb) products are highly heterogeneous due to post-translational modifications and other modifications that occur during manufacturing and storage. These modifications often result in charge variants that need to be analyzed using cation exchange chromatography.

Salt gradient and pH gradient methods have been used for charge variant analyses of proteins including monoclonal antibodies. Conventional salt and pH gradient methods cannot be directly coupled to MS due to high concentration of salt or the non-volatile nature of the mobile phase. MS compatible mobile phase systems have, due to their volatile nature, often suffer from poor stability, which leads to inconsistent pH gradients and retention time drift of chromatographic peaks.

A buffer preparation kit that is stable during shipping and storage while easy to assemble and use to generate MS compatible pH gradient mobile phases for the analysis of charge variants of biomolecules is herein described.

An MS compatible pH gradient mobile phase preparation kit for cation exchange chromatography of proteins including monoclonal antibodies is provided. A feature of this kit is to formulate and package the components required for the volatile, MS compatible mobile phases in separate containers to avoid degradation and change of the mobile phase during shipping and storage. The prepared mobile phase A is aqueous solution of a mixture of ammonium bicarbonate and acetic acid while mobile phase B is an aqueous ammonium hydroxide solution. The kit that was designed consists of containers A1 with ammonium bicarbonate and container A2 with acetic acid that is combined with MS grade water to generate mobile phase A. The kit also included container B1 that is used as is or diluted with MS grade water to generate mobile phase B. The containers are carefully chosen with regard to materials and design to allow for robust chemical compatibility and minimum leak/change of chemical components.

### Buffer Preparation System

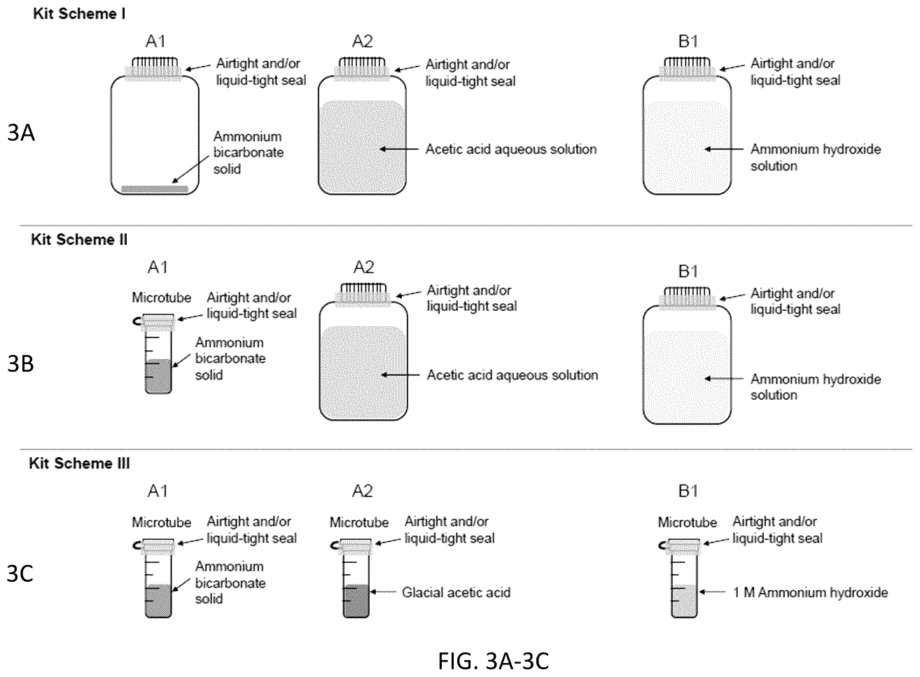

A buffer preparation system disclosed here consists of three separate kits (Kit I, Kit II, and Kit III) to make mobile phase A and B. These are depicted in FIGS. 3A, 3B and 3C. Mobile phase A is composed of 25 mM ammonium bicarbonate and 30 mM acetic acid with pH 5.30. Mobile phase B is composed of 10 mM ammonium hydroxide with pH 10.9.

### Container Compatibility

A feature of these mobile phase kits is to separate ammonium bicarbonate from acetic acid to minimize the loss of carbon dioxide during shipping and storage. The kit contains three parts, A1, A2 and B, which are used to store ammonium bicarbonate solution, acetic acid solution, and ammonium hydroxide solution, respectively. To further achieve the goal, each container was separately designed and chosen with different packaging materials to minimize the loss and change of the reagents during shipping and storage.

For ammonium bicarbonate solution, bottle materials are required to have low permeability for CO₂ to maintain the pH value of the solution and minimize the loss of CO₂. For instance, polyethylene terephthalate copolyester, glycol modified (PETG) serves as a good candidate for bottling A1 because of its lower permeability for CO₂. Media bottles made of durable, break-resistant PETG therefore were selected. A list of other candidate materials for bottling A1 is provided in Table 1. HDPE is considered less desirable for bottling A1 in view of the gas permeability as compared to ETFE, PETG or PMMA.

TABLE 1
Material candidates for bottling A1.

| | Permeability of CO2 | Permeability of CO2 |
|---|---|---|
| Material | (cc.-mil/100 in²-24 hr.-atm) | (cc.-mil/m²-24 hr.-bar) |
| ETFE | 250 | 97.14 |
| PETG | 125 | 48.57 |
| PMMA | 67.94 | 26.4 |
| HDPE | 580 | 225 |

For acetic acid solution and ammonia hydroxide solution, bottles made of high-density polyethylene (HDPE) which are excellent chemical resistant to most acid and base were used to allow maximum stability of the solution and minimize any leak of substances from bottles. A list of other candidate materials for bottling A2 and B is provided in Table 2 and 3

TABLE 2
Material candidates for bottling A2- E denotes
no damage after 30 days of constant exposure.

| Materials | 20° C. | 50° C. |
|---|---|---|
| LDPE | E | E |
| HDPE | E | E |
| PP | E | E |
| PPCO | E | E |
| PMP | E | E |
| FLPE | E | E |
| FEP | E | E |
| PFA | E | E |
| ETFE | E | E |
| PSF | E | E |

TABLE 3
Material candidates for bottling B- E denotes
no damage after 30 days of constant exposure.

| Materials | 20° C. | 50° C. |
|---|---|---|
| LDPE | E | E |
| HDPE | E | E |
| PP | E | E |
| PPCO | E | E |
| PMP | E | E |
| FEP | E | E |
| PFA | E | E |
| ETFE | E | E |

### Scheme for Kit I.

Kit I is depicted by FIG. 3A. A1 is a 250 ml bottle (with HPLC fitting neck) containing 0.395 g of solid of ammonium bicarbonate. A2 is a 250 ml bottle containing 200 mL of 30 mM acetic acid solution. B1 is a 250 mL bottle (with HPLC fitting neck) containing 200 mL of 10 mM ammonium hydroxide solution. To make mobile phase A, a 200 g solution from A2 is added into A1. A1 is then used as mobile phase A. B1 can be directly used as mobile phase B. Table 4 summarizes the Kit I, including instructions for its use. A PETG bottle is used for A1 while HDPE bottles are used for A2 and B1.

TABLE 4
Kit Scheme I.

| | Kit | | | |
|---|---|---|---|---|
| Kit Scheme I | component | Container | Contents | Instructions |
| Mobile Phase A | A1 | 250 mL | 0.395 g ammonium | 200 ml A2 solution is |
| | | bottle | bicarbonate solid | added into A1 to make |
| | A2 | 250 mL | 200 mL 30 mM | mobile phase A |
| | | bottle | acetic acid solution | |
| Mobile Phase B | B1 | 250 mL | 200 mL 10 mM | Directly used as |
| | | bottle | ammonium | mobile phase B |
| | | | hydroxide solution | |

### Scheme for Kit II.

Kit II is depicted by FIG. 3B. A1 is a microtube containing 0.395 g of solid ammonium bicarbonate. A2 is 250 ml bottle containing 200 mL of 20 mM acetic acid solution. B1 is a 250 ml bottle (with HPLC fitting neck) containing 200 mL of 10 mM ammonium hydroxide solution. To make mobile phase A, all the solids in A1 are added into the container holding A2. A1 is rinsed with A2 solution a few times to ensure complete transfer. A2 is then used as the mobile phase A. B1 is directly used as the mobile phase B. Table 5 summarizes the Kit II including instructions for its use.

TABLE 5
Kit Scheme II.

| | Kit | | | |
|---|---|---|---|---|
| Kit Scheme II | component | Container | Contents | Instructions |
| Mobile Phase A | A1 | microtube | 0.395 g ammonium | Solid in A1 is |
| | | | bicarbonate solid | completely transferred |
| | A2 | 250 mL | 200 mL 30 mM | into A2 to make |
| | | bottle | acetic acid solution | mobile phase A |
| Mobile Phase B | B1 | 250 mL | 200 mL 10 mM | Directly used as |
| | | bottle | ammonium | mobile phase B |
| | | | hydroxide solution | |

### Scheme for Kit III.

Kit III is depicted by FIG. 3C. A1 is a microtube containing 0.395 g of solid ammonium bicarbonate. A2 is a microtube and contains 0.360 mL of glacial acetic acid. B1 is a microtube containing 2 mL of 1M ammonium hydroxide solution. To make mobile phase A, the contents of A1 and A2 are mixed with 200 mL of MS grade water in an HPLC solvent bottle. A1 and A2 are each rinsed a few times to ensure complete transfer. To make mobile phase B, the contents of B1 are mixed with 200 mL MS grade water in an HPLC solvent bottle. B1 is rinsed a few times to ensure complete transfer. Table 6 summarizes the Kit III including instructions for its use.

TABLE 6
Kit Scheme III.

| | Kit | | | |
|---|---|---|---|---|
| Kit Scheme III | component | Container | Contents | Instructions |
| Mobile Phase A | A1 | microtube | 0.395 g ammonium | Combine all contents |
| | | | bicarbonate solid | in A1 and A2 with |
| | A2 | microtube | 0.360 mL glacial | 200 mL MS grade |
| | | | acetic acid | water in a larger |
| | | | | HPLC bottle to make |
| | | | | mobile phase A |
| Mobile Phase B | B1 | microtube | 2 mL of 1M | Mix all contents in B |
| | | | ammonium | with 200 ml MS |
| | | | hydroxide | grade water in a larger |
| | | | solution | HPLC bottle to |
| | | | | make mobile phase B |

The molar ratios of the components in Kits I-III were optimized to achieve an approximately linear pH gradient using these components. Running from 0% mobile phase B to 100% mobile phase B with a linear pump gradient generates a pH gradient from 5.30 to 10.90. Both mobile phase A and B are expected to remain stable and generate similar pH gradients for at least 24 hours. Fresh mobile phases should be prepared every 24 hours. The kits provide mobile phases that can be used with both weak cation exchange and strong cation exchange columns.

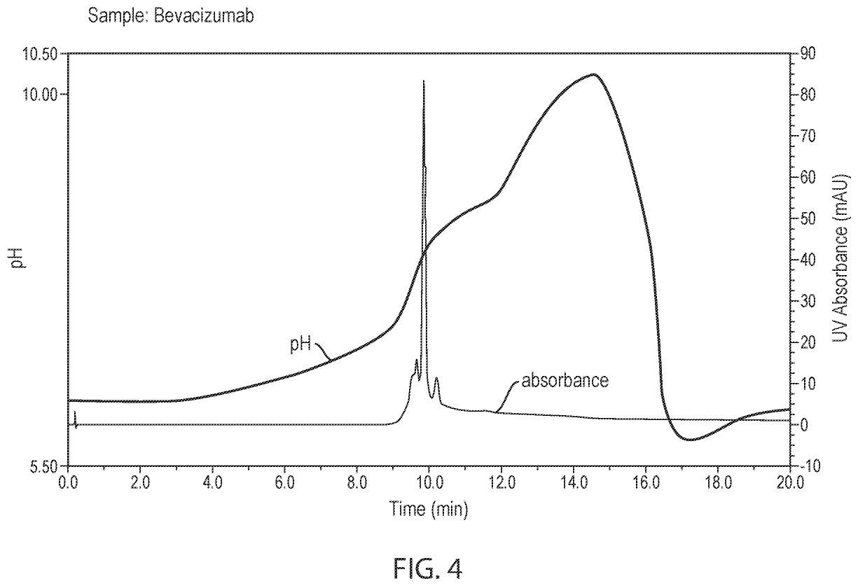

FIG. 4 shows a chromatogram of Bevacizumab, using a pH gradient from low to high pH. The pH vs time is also plotted. The Bevacizumab is detected by UV absorbance. The chromatography column can be coupled to a MS as well for analysis of Bevacizumab. Table 7 lists the conditions.

TABLE 7
Chromatography Conditions.

| | |
|---|---|
| Mobile phase A: | 25 mM ammonium bicarbonate, 30 mM |
| | acetic acid, pH 5.30 |
| Mobile phase B: | 10 mM ammonium hydroxide, pH 10.9 |
| Gradient: | 0-10 min: 0 to 100% B |
| | 10-13 min: 100% B |
| | 13.2-24 min: 0% B |
| Flow rate: | 0.4 mL/min |
| Column temperature: | 30° C. |
| Detection: | UV (280 nm) |
| Injection volume: | 1 μL |
| Sample: | mAb (5 mg/mL) |
| Column: | MAbPac ™ SCX-10 5 μm, 2.1 × 50 mm |

### Stability of Buffer System

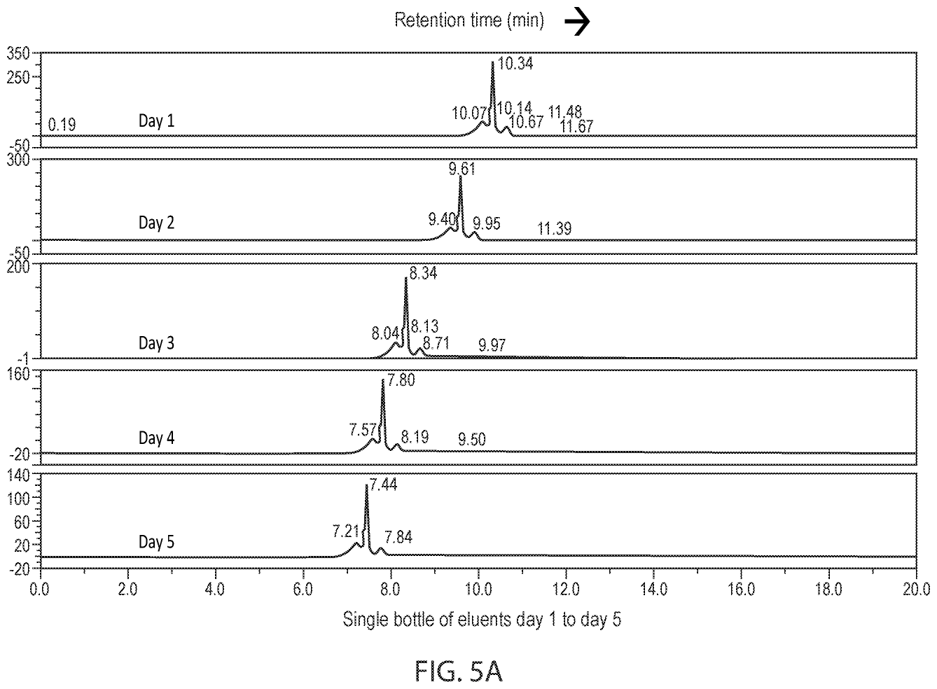

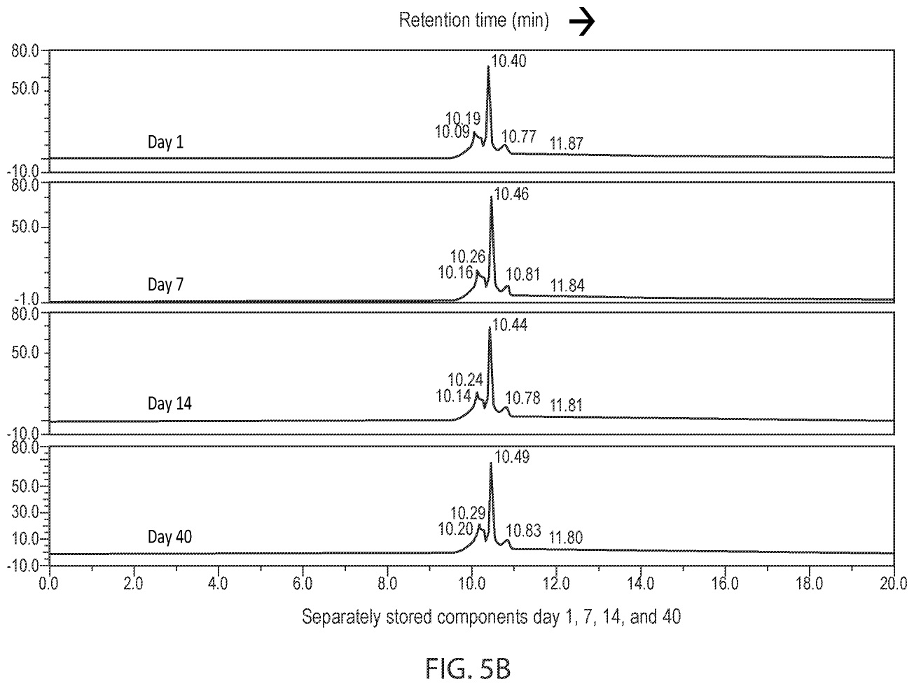

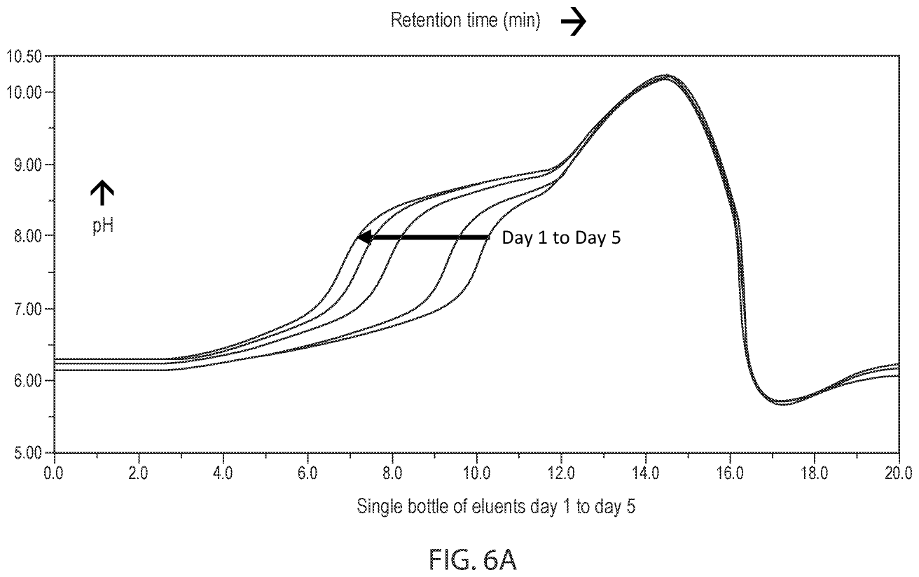

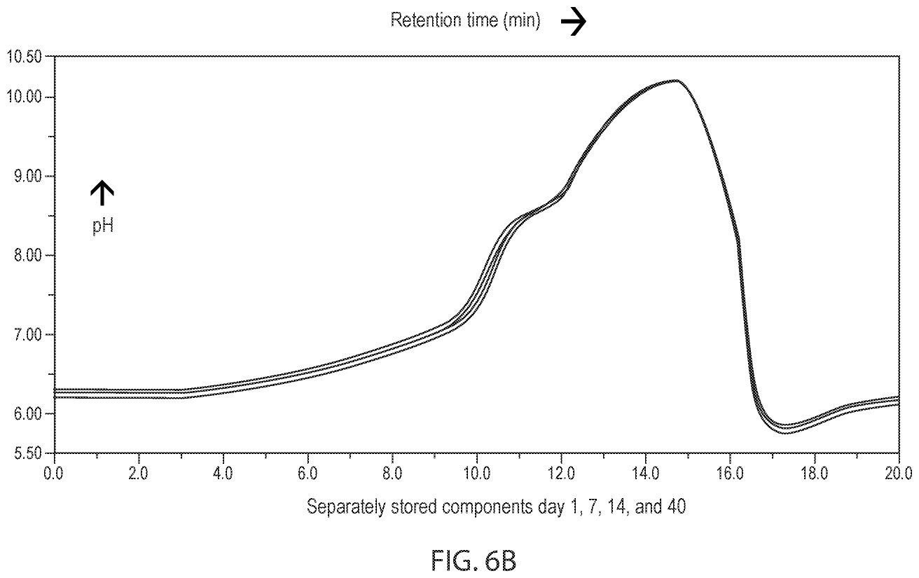

Comparative results for Bevacizumab separation with a MAbPac SCX-10 column shows that eluents prepared from the buffer preparation kit scheme I give high consistency in peak retention time and pH trace for at least 40 days, compared with single bottle of eluents, where peak retention time shifts from 10.34 min to 7.44 min within 5 days. This is illustrated with FIGS. 5A, 5B, 6A and 6B. FIG. 5A shows a drift in Bevacizumab peaks over five days when the Mobile Phase A components A1 and A2 are mixed on the first day. When the mobile phase A is prepared from components A1 and A2 each day, no significant drift of the Bevacizumab peaks is seen, as shown by FIG. 5B. FIG. 6A illustrates the change in the pH gradient using the mobile phase A where the components A1 and A2 are mixed on the first day. FIG. 6B in contrast shows the stability of the pH gradient when mobile phase A is prepared from components A1 and A2 each day.

### Instantaneous Mixing

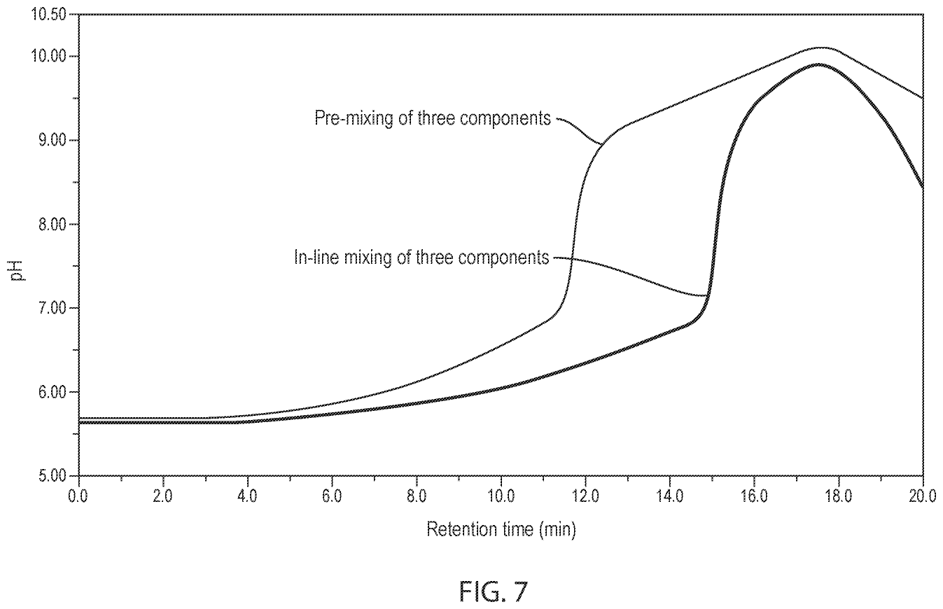

With the use of a quaternary pump, the eluent can also be generated in-situ from the three components A1, A2 and B. FIG. 7 shows the pH trace of pre-making eluent A and B with buffer kits and in-line mixing of buffer kits. In-line mixing shows some delay of increase in pH value but the slope at the turning point is similar to the pre-mixed eluent. This indicates that with the buffer preparation kit, eluent buffer can be generated in-situ and used directly.

### Buffer System not Using Volatile Components

A buffer system that included non-volatile components was made. Buffer A (pH 6) used 18 mM ammonium acetate, 5 mM 4-methylmorpholine, and 6 mM acetic acid. Buffer B (pH 10.3) used 12 mM acetic acid, 2 mM 4-methylmorpholine, 2 mM ammonium hydroxide, and 16 mM triethylamine. A similar gradient was used as listed in Table 7 and provided a linear pH trace. This was used with a ProPac Elite WCX column to analyze a sample containing Infliximab and Pertuzumab. Peaks, showing resolution of post translation modifications, were revolved by the UV detector. However, MS experiments were not successful due to MS contamination after several runs. It is speculated that, at least in part, this is due to 4-methylmorpholine which is not very volatile and can contaminate the MS.

Those having skill in the art, with the knowledge gained from the present disclosure, will recognize that various changes can be made to the disclosed apparatuses and methods in attaining these and other advantages, without departing from the scope of the present disclosure. As such, it should be understood that the features described herein are susceptible to modification, alteration, changes, or substitution. For example, it is expressly intended that all combinations of those elements and/or steps which perform substantially the same function, in substantially the same way, to achieve the same results are within the scope of the embodiments described herein. Substitutions of elements from one described embodiment to another are also fully intended and contemplated. The specific embodiments illustrated and described herein are for illustrative purposes only, and not limiting of that which is set forth in the appended claims. Other embodiments will be evident to those of skill in the art. It should be understood that the foregoing description is provided for clarity only and is merely exemplary. The spirit and scope of the present disclosure is not limited to the above implementation and examples but is encompassed by the following claims. All publications and patent applications cited above are incorporated by reference in their entirety for all purposes to the same extent as if each individual publication or patent application were specifically and individually indicated to be so incorporated by reference.

What is claimed is:

1. A pH gradient mobile phase preparation kit comprising:
a first component comprising a first reagent separate from a second reagent, wherein the first reagent, the second reagent, and a solvent together provide a volatile buffer solution having a low pH; and
a second component comprising a base, wherein the base combined with the solvent provide a volatile base solution having a high pH.

2. The kit according to claim 1, wherein the first reagent, the second reagent, the solvent and the base are volatile components.

3. The kit according to claim 1, wherein the volatile buffer solution is less stable than the first reagent separate from the second reagent.

4. The kit according to claim 1, wherein combining the first component with the second component in varied proportions provides a volatile mobile phase having a pH in a range between the low pH and the high pH.

5. The kit according to claim 1, wherein the first reagent is provided in an undiluted form and the second reagent is provided in a form diluted by the solvent.

6. The kit according to claim 1, wherein the first reagent is provided in an undiluted form and the second reagent is provided in an undiluted form, and the solvent is added to form the volatile buffer solution.

7. The kit according to claim 1, wherein the base is provided in an undiluted form.

8. The kit according to claim 1, wherein the base is ammonia or an amine.

9. The kit according to claim 1, wherein the second reagent is a carboxylic acid.

10. The kit according to claim 1, wherein the first reagent includes ammonia or an amine.

11. The kit according to claim 10, wherein the first reagent is ammonium bicarbonate.

12. The kit according to claim 1 including a first container containing the first reagent, a second container containing the second reagent, and a third container containing the second component, wherein the first container, the second container, and the third container are substantially gas impermeable and gas tight.

13. The kit according to claim 12, wherein the first container has a carbon dioxide gas permeability of less than 225 cc.-mm/m²-24 hr.-Bar.

14. The kit according to claim 1, wherein the solvent includes water.

15. A method of detecting one or more analytes comprised in a matrix of components in a sample, the method comprising:
injecting the sample into an injection valve, the injection valve being in fluid communication with a first end of a chromatography column;
pumping a volatile buffer solution having a low pH into the first end of the chromatography column, the volatile buffer solution comprising a first reagent and a second reagent provided from a kit;
pumping a volatile base solution having a high pH into the first end of the chromatography column, the volatile base solution including a base provided in the kit;
varying a proportion of the volatile buffer solution to the volatile base solution in the chromatography column by varying corresponding amounts of the volatile buffer solution and the volatile base solution pumped to the first end of the chromatography column, thereby providing a volatile mobile phase with a pH time-gradient flowing through the chromatography column;
eluting the sample through the chromatography column in the volatile mobile phase thereby separating the analyte from the matrix components;
injecting the sample into a mass spectrometer to provide a mass spectrum of the one or more analytes.

16. The method according to claim 15, wherein the first reagent and the second reagent from the kit are combined to form the volatile buffer solution less than 10 days prior to pumping the volatile buffer solution into the first end of the chromatography column.

17. The method according to claim 15, wherein the mass spectrometer includes an electrospray ionization source to create an aerosol of the analyte and the volatile mobile phase.

18. The method according to claim 15, wherein the first reagent includes ammonium bicarbonate, and the second reagent includes acetic acid.

19. The method according to claim 15, wherein the chromatograph column is an ion exchange column.

20. The method according to claim 15, wherein the analyte is a biomolecule having a pI between pH 4 and pH 10.

21. The method according to claim 15, wherein the pH time-gradient includes a positive gradient, wherein the pH increases with time while separating the analyte from the matrix components.

22. The method according to claim 15, wherein the first reagent and the second reagent from the kit are combined in-line to form the volatile buffer solution.
